(19) United States
(12) Patent Application Publication
(10) Pub. No.: US 2015/0342990 A1
(43) Pub. Date: Dec. 3, 2015

(54) METHODS AND COMPOSITIONS FOR THE USE OF SILVER TO PREVENT AND TREAT ACNE

(71) Applicant: METABEAUTY, INC., MIAMI, FL (US)

(72) Inventor: LESLIE BAUMANN, MIAMI, FL (US)

(73) Assignee: METABEAUTY, INC, MIAMI, FL (US)

(21) Appl. No.: 14/725,832

(22) Filed: May 29, 2015

Related U.S. Application Data
(60) Provisional application No. 62/004,819, filed on May 29, 2014.
(60) Provisional application No. 62/057,173, filed on Sep. 29, 2014.

(51) Int. Cl. A61K 33/38; A61K 45/06; A61K 31/07; A61K 9/70; A61K 31/327

(52) U.S. Cl. CPC A61K 33/38; A61K 9/70; A61K 31/327; A61K 31/07; A61K 45/06

(57) ABSTRACT

Embodiments herein describe skincare compositions having colloidal silver, silver textiles coated with colloidal silver and acne treatment ingredient nanoparticles, methods of treating and preventing acne, rosacea, and other inflammatory lesions using such skincare compositions and textiles, methods of treating skin and preventing acne after resurfacing modalities using such skincare compositions and textiles, and kits for such methods. Embodiments also describe regimens for acne treatment and prevention using the described skincare compositions, textiles, and acne treatment compositions.

### RELATED APPLICATIONS

This application claims the benefit U.S. Provisional Application No. 62/004,819, filed May 29, 2014, and U.S. Provisional Application No. 62/057,173, filed Sep. 29, 2014, the disclosure of each of which is hereby incorporated herein by reference in its entirety.

### BACKGROUND

Free silver ions, or radicals, are known to be the active agents of antimicrobial silvers. Of interest are their extremely potent antimicrobial properties, as only one part per 100 million of elemental silver is an effective antimicrobial in a solution. It is believed that silver ions kill micro-organisms instantly by blocking the respiratory enzyme system (energy production), while having no negative effect on human cells. No known bacterial resistance has been reported to the silver ion, unlike current antibiotics.

Although silver itself has been shown to be harmless to normal human tissue, there has been toxicity noted from the salt or complexes used to deliver the silver. Silver nitrate solutions used on burns and wounds are toxic to new cells on the wound surface because nitrate is a potent oxidizing agent. Likewise, the sulfadiazine component of silver sulfadiazine produces local and systemic (bone marrow) damage and potent pro-inflammatory properties increasing wound surface exudates. Orally ingested silver may cause argyria, and some forms of silver may lead to contact dermatitis. For example, the AgNO₃ form of silver undergoes a reduction of AG⁺ to Ag⁰ which may cause the wound to blacken and may result in tattooing of the skin.

Acne is a group of diseases whose initial pathology is the comedone and includes acne vulgaris, neonatal acne, infantile acne, acne cosmetica, and pomade acne. It is estimated that 85% of adolescents are affected by acne. Adolescents, however, are often noncompliant with any medical treatment/regimen prescribed by a physician. In the case of acne, general non-compliance is exacerbated by sun sensitivity, GI upset, dryness, redness, and skin sensitivity experienced when using many of the most widely-prescribed acne treatment products. There is also a growing number of patients who develop acne in their late twenties or thirties. It is estimated that about 25% of men and about 50% of women will be affected by acne at some point during their adult lives.

Acne is characterized by pimples, pustules, deeper lumps (cysts or nodules), and plugged pores (blackheads and whiteheads), that occur on the face, neck, chest, back, shoulders, and even the upper arms. Acne lesions are also seen in skin diseases such as rosacea, folliculitis, and shaving irritation (pseudofolliculitis barbae). Acne is also seen in approximately 30% of post skin resurfacing patients—particularly those treated with Fraxel laser resurfacing. It is also commonly seen after medical facials, which have been reported to cause acne in 50% of people who receive a facial. The cause of acne seen after resurfacing procedures is poorly understood but is believed to be due to disruption of the keratinization process during the resurfacing procedure. The deeper resurfacing methodologies are associated with a higher amount of post procedure acne than the superficial resurfacing modalities such as superficial chemical peels and intense pulsed light.

There has been and remains a long-felt, but as yet unmet need, for an effective, easily followed method and/or regimen for the treatment, prevention, and management of acne and post-procedural and post-wound inflammatory lesions, pigmentation, and scarring. This need is met by the compositions, textiles and methods of embodiments described herein.

### BRIEF SUMMARY

Embodiments herein are directed to a skincare composition comprising colloidal silver (or a derivative thereof) and at least one hydroxy acid, wherein the composition has a pH of up to about 5. In some embodiments, the skincare composition comprises (a) an amphoteric surfactant and/or a zwitterionic surfactant having an isoelectric point of pH 7 or below; (b) a non-ionic surfactant; (c) colloidal silver; (d) an acid to complex with the colloidal silver, wherein the molar ratio of the acid to the colloidal silver (c) is at least 1:1; and (e) one or more hydroxy acid(s). Some embodiments herein are directed to a skincare composition comprising (a) an amphoteric surfactant and/or a zwitterionic surfactant having an isoelectric point of pH 7 or below; (b) a non-ionic surfactant; (c) a complex formed by colloidal silver and an acid; and (d) one or more hydroxy acid(s). In some embodiments, the amphoteric surfactant may be an ampholyte. In some embodiments, the non-ionic surfactant may be a polyglucoside. In some embodiments, the hydroxy acid is salicylic acid.

Embodiments herein are also directed to a skincare composition comprising colloidal silver (or a derivative thereof) and at least one acne treatment ingredient. In some embodiments, the acne treatment ingredient may be benzoyl peroxide, clindamycin, erythromycin, polyhydroxy acid, willowbark extract, salicylic acid, resorcinol, resorcinol monoacetate, niacinamide, sulfur, beta carotene, vitamin A or derivatives thereof, retinol, tretinoin, adapalene, tazarotene, retinyl palmitate, retinyl linoleate, other ingredients that bind Toll-like 2 receptors, or a combination thereof. In some embodiments, the acne treatment ingredient may be benzoyl peroxide, a retinoid, or salicylic acid. In some embodiments, the skincare composition may be a cleanser, a toner, an ointment, a gel, a cream, a lotion, a solution, or a combination thereof.

Embodiments herein are directed to a skincare kit for treatment of acne comprising a silver textile, a skincare composition, and an acne treatment composition, wherein the acne treatment composition comprises an active selected from benzoyl peroxide, clindamycin, erythromycin, polyhydroxy acid, willowbark extract, salicylic acid, resorcinol, resorcinol monoacetate, sulfur, beta carotene, vitamin A or derivatives thereof, retinol, tretinoin, adapalene, tazarotene, retinyl palmitate, retinyl linoleate, other ingredients that bind Toll-like 2 receptors, or a combination thereof. In some embodiments, the components (e.g. benzoyl peroxide, salicylic acid, silver, zinc, etc.) described herein as being in nanoparticle form may be in the form of micro particles (greater than 100 nanometers, but smaller than one micron), micron sized particles, microencapsulated materials (encapsulated materials that may be engineered to rupture by friction, UV light, be soluble in water or body fluids (blood, sweat, or other body fluids), be temperature sensitive, etc.), matrices (materials mixed with polymers, proteins, pectin, gelatin, preservatives or other materials to create matrices that provide controlled delivery, efficacy, and longevity), materials coated with polymers, proteins, pectin, gelatin, preservatives or other materials to achieve controlled delivery, efficacy, and longevity, microspheres (actives incorporated into components that are emulsified prior to application—in one example, actives are blended into waxes or other non-water soluble materials that have low melt temperatures and emulsified to make aqueous emulsions at temperatures that cause the carrier (waxes or other inert carriers) to melt while being vigorously agitated in an aqueous solution containing specific surfactants. This creates micro-spheres of emulsified products in an aqueous bath. The emulsion may be applied to textiles or included in gels, lotions, creams or sprays), smart materials including polymers that swell, shrink or provide other characteristics as a response to environmental stimuli (moisture, pH, temperature, etc.), UV curable materials (e.g. UV curing may be used to create durability), low temperature melt materials (e.g. spheres of low temperature melt materials may be applied on the textiles and heated to form films on the textile after application), or a combination thereof.

In some embodiments, the skincare composition may be a cleanser, a toner, an ointment, a gel, a cream, a lotion, a solution, or a combination thereof. In some embodiments, the skincare composition may include comprises (a) an amphoteric surfactant and/or a zwitterionic surfactant having an isoelectric point of pH 7 or below; (b) a non-ionic surfactant; (c) colloidal silver; (d) an acid to complex with the colloidal silver, wherein the molar ratio of the acid to the colloidal silver (c) is at least 1:1; and (e) one or more hydroxy acid(s). In some embodiments, the skincare composition comprises (a) an amphoteric surfactant and/or a zwitterionic surfactant having an isoelectric point of pH 7 or below; (b) a non-ionic surfactant; (c) a complex formed by colloidal silver and an acid; and (d) one or more hydroxy acid(s). In some embodiments, the skincare composition may be in the form of a wipe. In some embodiments, the acne treatment composition comprises retinoid nanoparticles, benzoyl peroxide nanoparticles or a combination thereof. In some embodiments, the retinoid may be selected from retinol, retinyl linoleate, retinyl palmitate, adapalene, tretinoin, tazarotene, or a combination thereof. In some embodiments, the silver textile is a pillowcase. In some embodiments, the silver textile is a shirt.

Embodiments herein are also directed to a skincare regimen for treating acne, the regimen comprising: washing the affected area with a cleanser of embodiments herein, applying a toner of embodiments herein to the affected area; and contacting the affected area with a silver textile of embodiments herein for at least about one hour. In some embodiments, the skin care regimen for treating acne may further include applying an acne treatment composition comprising nanoparticles of an active to the affected area. In some embodiments, the acne treatment composition may be applied only to acne lesions (spot treatment) or may be applied to the entire affected area, such as the face, back or chest. Some embodiments herein are directed to a skin care regimen to treat acne, the regimen comprising a morning routine of applying a toner of embodiments herein to the affected area, and an evening routine of washing the affected area with a cleanser of embodiments herein, and contacting the affected area with a silver textile of embodiments herein for at least about one hour. In some embodiments, the evening routine may further include applying an acne treatment composition of embodiments herein. In some embodiments, the acne treatment composition may be applied to the lesions or to the entire affected area. In some embodiments, the affected area has mild to moderate acne, moderate acne, moderate to severe acne, or severe acne. Some embodiments are directed to a skin care regimen to treat acne, the regimen comprising a morning routine including washing the affected area with a cleanser of embodiments herein, and applying a toner of embodiments herein to the affected area; and an evening routine including washing the affected area with a cleanser of embodiments herein, applying a toner of embodiments herein, applying an acne treatment composition of embodiments herein, and contacting the affected area with a silver textile of embodiments herein for at least one hour.

Embodiments herein are directed to methods of using the skincare compositions and/or textiles of embodiments herein to treat acne, rosacea, other inflammatory lesions, post-inflammatory pigmentation, or a combination thereof. In some embodiments, a method of treating acne comprises contacting an affected area of skin with a silver textile of embodiments herein. In some embodiments, the silver textile may also comprise zinc. These textiles may be further coated with other metals or other anti-inflammatory ingredients, including, without limitation, calendula, chamomile, benzoyl peroxide, niacinamide, salicylic acid, NSAIDS, and other ingredients that block the arachidonic acid cascade. In some embodiments, the silver textile may be a skin patch, cloth (both disposable and reusable), pillowcase, bed sheet, mask, shirt, t-shirt, tank top, pajamas, and the like. In some embodiments, the silver may be colloidal silver.

In some embodiments, the method further comprises topically administering a skincare composition of embodiments herein. In some embodiments, the method comprises topically administering the skincare composition before contacting the affected area with a silver textile of embodiments herein. In some embodiments, the method comprises topically administering the skincare composition after contacting the affected area with a silver textile of embodiments herein. In some embodiments, the skincare composition may comprise salicylic acid, benzoyl peroxide, tretinoin, calendula, chamomile, NSAIDS, other ingredients that block the arachidonic acid cascade, or a combination thereof.

In some embodiments, the skincare composition comprises at least one hydroxy acid. In some embodiments, the at least one hydroxy acid is salicylic acid, willowbark extract, poly hydroxyacid, tannic acid, hydroxybenzoic acid, juniperic acid, tartaric acid, phytic acid, glycolic acid, lactic acid, citric acid, or a combination thereof. In some embodiments, the skincare composition may further include other metals, such as, but not limited to, alloy, copper, zinc, nickel, titanium, cobalt, gold, iron, chromium, platinum, plutonium, mercury, nickel, magnesium, cadmium, gadolinium, lithium, tungsten, or a combination thereof.

In some embodiments, a method of treating inflammatory lesions comprises contacting an affected area of skin having inflammatory lesions or the area of inflamed skin with a skincare composition and/or silver textile of embodiments herein. In some embodiments, the method further comprises topically co-administering an anti-inflammatory composition. In some embodiments, the anti-inflammatory composition comprises at least one hydroxy acid. In some embodiments, the at least one hydroxy acid is salicylic acid, willowbark extract, poly hydroxyacid, tannic acid, hydroxybenzoic acid, juniperic acid, tartaric acid, glycolic acid, lactic acid, citric acid, or a combination thereof. In some embodiments, the anti-inflammatory composition may further include other anti-inflammatory ingredients, including, without limitation, calendula, chamomile, NSAIDS, and other ingredients that block the arachidonic acid cascade.

In some embodiments, a method of treating rosacea lesions comprises contacting an affected area of skin having rosacea lesions or the area of rosacea prone skin with the skincare compositions and/or silver textile of embodiments herein. In some embodiments, the method further comprises topically co-administering a rosacea treatment composition. In some embodiments, the rosacea treatment composition comprises at least one hydroxy acid. In some embodiments, the at least one hydroxy acid is salicylic acid, willowbark extract, poly hydroxyacid, tannic acid, hydroxybenzoic acid, juniperic acid, tartaric acid, glycolic acid, lactic acid, citric acid, or a combination thereof. In some embodiments, the rosacea treatment composition may further include other anti-inflammatory ingredients, including, without limitation, calendula, chamomile, NSAIDS, and other ingredients that block the arachidonic acid cascade.

Some embodiments are directed to a method of treating lesions after skin resurfacing modalities, such as, without limitation, laser or chemical peels, comprising contacting an affected area of skin having lesions or the area treated with a skin resurfacing modality with the silver containing skincare compositions and/or silver textiles of embodiments herein. In some embodiments, the method further comprises topically coadministering a treatment composition comprising at least one hydroxy acid. In some embodiments, the at least one hydroxy acid is salicylic acid, willowbark extract, poly hydroxyacid, tannic acid, hydroxybenzoic acid, juniperic acid, tartaric acid, glycolic acid, lactic acid, citric acid, or a combination thereof. In some embodiments, the treatment composition may further include other anti-inflammatory ingredients, including, without limitation, calendula, chamomile, NSAIDS, and other ingredients that block the arachidonic acid cascade.

Some embodiments are directed to a method of treating post-inflammatory hyperpigmentation in a subject comprising contacting the affected skin of the subject with a silver containing skincare compositions and/or silver textile described in embodiments herein. Some embodiments are directed to a method of preventing the onset of post-inflammatory hyperpigmentation in a subject comprising contacting the affected skin of the subject with a skincare composition and/or silver textile described in embodiments herein. In some embodiments, the skincare composition and/or silver textile of embodiments herein may be used to prevent the post-inflammatory hyperpigmentation associated with acne, laser, rosacea or any other inflammatory process.

Some embodiments are directed to a method of treating skin after skin resurfacing modalities, the method comprising contacting the affected skin of the subject with a silver containing skincare composition and/or silver textile described in embodiments herein wherein the method speeds healing of the skin, reduces acne, and decreases the occurrence of post-inflammatory pigmentation.

Some embodiments are directed to a method of treating a pigmentation disorder in a subject comprises contacting an affected skin of the subject with a silver containing skincare compositions and/or textile described in embodiments herein. In some embodiments, the pigmentation disorders may be selected from melasma, age spots, solar lentigos, hyperpigmentation, lentigines, post-inflammatory pigmentation, pityriasis versicolor, phytophotodermatitis, poikiloderma of Civatte, ephelides (freckles), vitiligo, or a combination of any of foregoing. In some embodiments, the skincare composition and/or silver textile can be administered in combination with a topical pigmentation disorder treatment. In some embodiments, the topical pigmentation disorder treatment may include a tyrosinase inhibitor, lignin peroxide, anti-oxidant, hydroxy acid, PAR-2 blocker, or a combination thereof. In some embodiments, the silver textile may include a tyrosinase inhibitor, lignin peroxide, anti-oxidant, hydroxy acid, PAR-2 blocker, or a combination thereof.

Some embodiments are directed to a method of accelerating healing of skin after a medical facial in a subject in need thereof, the method comprising contacting an area of skin treated with the medical facial with the skincare composition and/or silver textile of embodiments herein, wherein the medical facial comprises the steps of (1) resurfacing an affected area of skin of the subject to form resurfaced skin; (2) topically applying to the resurfaced skin an effective amount of an imidazoquinoline amine derivative to form imidazoquinoline amine derivative treated skin; and (3) topically applying to the imidazoquinoline amine derivative treated skin an effective amount of a retinoid and/or an antioxidant. In some embodiments, the medical facial is as described in U.S. patent application Ser. No. 12/403,249, which is incorporated herein by reference in its entirety.

Some embodiments are directed to a method of treating acne comprising applying an exfoliator to an affected area and contacting the affected area with the silver textile of embodiments herein. In some embodiments, the exfoliator may be a skincare composition comprising alpha hydroxy acid, salicylic acid, or a combination thereof. It is believed that combining the use of an exfoliator decreases the thickness of the stratum corneum and increases smoothness of the skin's surface allowing better contact of the skin with the silver textile. In some embodiments, the exfoliator may be a chemical exfoliator, such as an alpha hydroxyl acid or a salicylic acid, and/or a mechanical exfoliator, such as a brush, a bead, a loofa, or microdermabrasion.

Some embodiments are directed to a method of treating acne comprising applying to an affected area of skin an antimicrobial product intended to kill P. acnes; and contacting the affected area with a silver textile of embodiments herein. In some embodiments, the silver textile further includes zinc. In some embodiments, the antimicrobial product may be selected from clindamycin or doxycycline. In some embodiments, the antimicrobial product is benzoyl peroxide (BP). It is believed that benzoyl peroxide increases oxidation of the silver leading to an increased release of silver ions. It is further believed that these positively charged silver ions bind the negatively charged bacteria and kill it. In some embodiments, the antimicrobial product may be formulated as a cleanser intended to be rinsed off, or as a solution, a gel or a lotion intended to be left on the skin. In some embodiments, the antimicrobial product may be selected from blue light (410-495 nm). In some embodiments, the blue light may be used as a hand held device, or as a light emitted from a mobile device such as a cell phone.

### DETAILED DESCRIPTION

Before the present silver containing skincare compositions, silver textiles, and methods are described, it is to be understood that this invention is not limited to the particular processes, textiles, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety.

As used in the present application, the following terms having meanings set out in the Final Monograph for Topical Acne Drug Products for Over-the-Counter Human Use as promulgated by the Food and Drug Administration in Volume 56, Number 159 of the Federal Register at pages 41008-41020.

By the term “acne” is meant a disease involving the oil glands and hair follicles of the skin that is manifested by blackheads, whiteheads, acne pimples, and acne blemishes, each as defined below.

By the term “acne blemish” is meant a visible flaw in the skin resulting from acne.

By the term “acne pimple” is meant a small prominent, inflamed elevation of the skin resulting from acne. The skilled artisan will understand that papules (closed, red bumps surrounded by skin inflammation) and pustules (bumps characterized by a central white or yellow dot surrounded by inflammation) are often referred to as “pimples”.

Hypercornification of the pilosebaceous ducts can be seen histologically as microcomedones, and clinically as “blackheads” and “whiteheads”. By “blackhead” is meant a condition of the skin that occurs in acne and is characterized by a black tip. By “whitehead” is meant a condition of the skin that occurs in acne and is characterized by a small, firm, whitish elevation of the skin.

The skilled artisan will understand that the term “comedone” is used to describe a non-inflammatory acne lesion, including blackheads and whiteheads. Comedones occur when a skin pore is “clogged” or “blocked” by a combination of sebum plugs (secreted by a pilosebaceous gland), cell debris, and microorganisms (especially the bacterium Propionibacterium acnes, abbreviated as P. acnes). Comedones may be open (blackheads), with the upper, visible portion being darkened by oxidation. Comedones may also be closed (whiteheads), not reaching the surface to be extruded. Closed comedones may become pustules and/or deep inflammatory lesions.

Acne blemishes, acne pimples, whiteheads, blackheads and comedones are sometimes referred to in the present application collectively as “acne lesions.” The term acne lesions shall also include includes acne vulgaris, neonatal acne, infantile acne, pomade acne, cystic acne, nodulocystic acne, acne cosmetica, folliculitis, psudeofolliculitis barbae, and rosacea or a combination thereof.

As used in the present application, the term “acneic skin” is to be understood to mean skin that exhibits acne lesions, and includes the term “acne-prone skin,” which is to be understood to mean skin that has a propensity to develop acne lesions.

It must also be noted that as used herein and in the appended claims, the singular forms “a”, “an”, and “the” include plural reference unless the context clearly dictates otherwise. Thus, for example, reference to a “antioxidant” is a reference to one or more antioxidants and equivalents thereof known to those skilled in the art, and so forth.

As used herein, the term “about” means plus or minus 10% of the numerical value of the number with which it is being used. Therefore, about 50% means in the range of 45%-55%.

The term “inhibiting” includes the contacting of one or more areas of a subject's skin with a silver containing skincare composition and/or silver textile of the present invention to prevent the onset of the symptoms, alleviating the symptoms, or eliminating the disease, condition or disorder.

The term “metallize” refers to the process of adding a metal to a textile.

As used herein, the term “rosacea lesions” refers to papules, pustules and erythema associated with rosacea.

Unless otherwise indicated, the term “skin” means that outer integument or covering of the body, consisting of the dermis and the epidermis and resting upon subcutaneous tissue.

As used herein, the term “therapeutic” means an agent utilized to treat, combat, ameliorate, prevent or improve an unwanted condition or disease of a patient. In part, embodiments of the present disclosure are directed to the treatment of acne lesions, including, without limitation, (a) helping to clear and/or dry acne lesions, (b) reducing the number and/or severity of acne lesions, (c) helping keep the skin clear of new acne lesions and/or (d) helping to prevent the development of new acne lesions.

As used herein, the term “textile” refers to a wide variety of material including paper, natural or synthetic fibers, threads and yarns made from materials such as cotton, rayon, wool, jute, polyesters, polyamide, aromatic polyamides, para-aramides (Kevlar®), meta-aramides (Nomex®), glass fibers or pure silica, nylon, polyacetates, polyacrylics as well as cellulosics in general, or a combination thereof. More particularly, the term refers to fibers woven into a fabric such as knitting, and non-woven hydrophilic fabrics or webbing used in anti-pathogenic applications such as in the medical field, hospitals, biotechnology and food and dairy processing. Exemplary textile products of this genre include skin patches, cloth (both disposable and reusable) including, without limitation, tank tops, t-shirts, pants, shorts, underwear, pillowcases, bed sheets, masks, shirts, pajamas, and the like.

As used herein, the term “silver textile” refers to a textile coated or woven or integrated with a therapeutically-effective amount of silver.

As used herein, the term “yarn” refers to a continuous often plied strand composed of either natural or man-made fibers or filaments that may be used in non-woven fabrics and in weaving and knitting to form a fabric. The term “yarn” may be used interchangeably with “fiber” or “thread.”

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects, such as skin coloration or pain. With reference to the step of embodiments described herein in which a skin treatment composition is used, in some embodiments, “treating” means applying the composition to the skin for at least a specified period of time, in accordance with the directions supplied with the product or as directed by a doctor or aesthetician, and either rinsing the formulation off the skin or leaving the formulation on the skin.

Using the skincare composition and/or silver textile of embodiments herein may prevent and treat acne, rosacea lesions, inflammatory lesions, scarring, and/or pigmentation, including acne, inflammatory lesions, scarring and/or pigmentation seen after skin resurfacing modalities, and may also speed the wound healing process.

The treatment regimen described herein may be adjusted by the clinician according to methods known to the clinician in order to obtain the optimal clinical response. A treatment regimen may be decided based on the characteristics of the subject being treated, e.g., the particular animal treated, age, weight, health, concurrent diagnoses, types of concurrent treatment, if any, and frequency of treatments, and can be easily determined by one of skill in the art (e.g., by the clinician).

The methods, textiles, and compositions of embodiments described herein provide several clinically significant benefits to patients having acneic skin or acne-prone skin including one or more of (a) helping to clear and/or dry acne blemishes, acne pimples, blackheads and/or whiteheads, (b) reducing the number and/or severity of acne blemishes, acne pimples, blackheads and/or whiteheads, (c) helping keep the skin clear of new acne blemishes, acne pimples, blackheads and/or whiteheads and/or (d) helping to prevent the development of new acne blemishes, acne pimples, blackheads and/or whiteheads. Further, certain embodiments describe using the silver containing skincare compositions and/or silver textile of embodiments herein may prevent the inflammatory lesions, scarring, and/or pigmentation seen after skin resurfacing modalities. In addition, the methods, textiles and compositions described herein speed the wound healing process. The inventive methods of embodiments described herein are also directed to reducing the number and/or severity of acne blemishes, acne pimples, blackheads and/or whiteheads, which further allows the skin to heal.

Similar benefits are seen in patients having other inflammatory lesions such as rosacea,

In some embodiments, acne lesions can be graded by the grading scale proposed in C. H. Cook et al., Arch. Dermatology, 571 -575 (1979). In some embodiments, acne lesions may be classified as mild (0-2 on the Cook scale), mild to moderate (3 on the Cook scale), moderate (4-5 on the Cook scale), moderate to severe (6-7 on the Cook scale), or severe (8 on the Cook scale).

Embodiments described herein are directed to novel skincare formulations (e.g cleanser and/or toner) comprising colloidal silver (or a derivative thereof) and at least one hydroxy acid, wherein the composition has a pH of up to about 5. In some embodiments, the at least one hydroxy acid is salicylic acid. Without wishing to be bound to a particular theory, the inventor believes the clinical efficacy of these compositions is attributable to the ability of the compositions to penetrate skin pores in acneic and acne-prone skin, thereby controlling, reducing the number of, clearing and/or eliminating most acne blemishes, acne pimples, blackheads, and/or whiteheads. Embodiments described herein are also directed to novel skincare formulations (e.g cleanser and/or toner) comprising colloidal silver (or a derivative thereof) and at least one acne treatment ingredient, wherein the acne treatment ingredient is benzoyl peroxide, clindamycin, erythromycin, polyhydroxy acid, willowbark extract, salicylic acid, resorcinol, resorcinol monoacetate, niacinamide, sulfur, beta carotene, vitamin A or derivatives thereof, retinol, tretinoin, adapalene, tazarotene, retinyl palmitate, retinyl linoleate, other ingredients that bind Toll-like 2 receptors, or a combination thereof.

Some embodiments are directed to a method for the treatment and management of acne where the method comprises the steps of (i) treating acneic skin or acne-prone skin with a skincare composition comprising colloidal silver (or a derivative thereof) and at least one hydroxy acid, wherein the formulation has a pH of up to about 5, and (ii) contacting the acneic skin or acne-prone skin with a textile containing a therapeutically-effective amount of silver.

Some embodiments are directed to methods for the treatment and management of skin conditions related to acne and known in the art as “inflammatory dermatoses”, which may include inflammatory acne, erythematotelangiectatic rosacea, and papulopustular rosacea. In some embodiments, a method of treating or managing inflammatory dermatoses comprises the steps of (i) treating the inflammatory dermatoses with at least one of a skincare composition of embodiments herein that contains colloidal silver (or a derivative thereof), where the formulation preferably has a pH of lower than 5, and also contains at least one hydroxyacid and (ii) contacting the inflammatory dermatoses with a textile containing a therapeutically-effective amount of silver.

In some embodiments, a method of treating or managing inflammatory dermatoses comprises contacting the inflammatory dermatoses affected skin with a textile comprising a therapeutically effective amount of silver. In some embodiments, a method of treating or managing acneic skin or acne prone skin comprises contacting the inflammatory dermatoses affected skin with a textile comprising a therapeutically effective amount of silver.

By “inflammatory acne” is meant a dermatologic condition in which multiple inflammatory lesions as well as comedones and papules/pustules are present. This condition may or may not also present with small nodulocystic lesions.

By “erythematotelangiectatic rosacea” is meant a type of rosacea characterized by flushing and persistent central facial erythema. Telangiectasias, visibly dilated blood vessels on the skin surface, are common in this condition.

By “papulopustular rosacea” is meant a dermatological condition characterized by persistent central facial erythema with transient papules, pustules, or both in a central facial distribution.

The presentation of the erythematotelangiectatic and papulopustular subtypes of rosacea are described in Wilkin et al., J. Am. Acad. Dermatol., pp. 907-912 (June 2004). These subtypes of rosacea can be, and preferably is, graded by the standard grading system disclosed in Wilkin, et al.

Some embodiments are directed to methods for the treatment and management of folliculitis, a dermatologic condition that is often clinically indistinguishable from acne. In folliculitis, the hair follicle is blocked by dyskeratinization, bacteria accumulates in the blocked follicle, and lesions, similar in appearance to acne lesions, develop, often with surrounding areas of inflammation. This aspect of the invention is also directed to the treatment and management of pseudo-folliculitis barbae, a condition where shaven hair curls into the skin and becomes imbedded. As in acne and folliculitis, the blocked pore/follicle accumulates bacteria, and becomes infected, which is often accompanied by inflammation of the areas of skin surrounding the imbedded hair. In some embodiments, a method of treating folliculitis and/or pseudofolliculitis barbae comprises the steps of (i) treating the folliculitis/pseudofolliculitis barbae with a skincare composition of embodiments herein that comprises colloidal silver (or a derivative thereof), and at least one acne treatment ingredient; and (ii) contacting skin exhibiting folliculitis/pseudofolliculitis barbae with a textile containing a therapeutically-effective amount of silver. In some embodiments, the formulation has a pH of up to about 5.

In some embodiments, a method of treating folliculitis and pseudofolliculitis barbae comprises contacting the folliculitis and/or pseudofolliculitis barbae affected skin with a textile containing a therapeutically effective amount of silver.

In some patients having one or more of acneic skin, acne-prone skin, inflammatory dermatoses, folliculitis and/or pseudofolliculitis barbae, the clogged pores can have an enlarged appearance. Accordingly, some embodiments described herein are directed to reducing the visible appearance of pores by (i) treating the skin in need thereof with a skincare composition of embodiments herein that comprises colloidal silver (or a derivative thereof), and at least one acne treatment ingredient; and (ii) contacting the skin with a textile containing a therapeutically-effective amount of silver.

Some embodiments are directed to treating skin after resurfacing procedures by (i) treating the skin in need thereof with at least one of a cleanser or toner formulation that comprises colloidal silver (or a derivative thereof), and at least one acne treatment ingredient; and (ii) contacting the skin with a textile containing a therapeutically-effective amount of silver. Resurfacing procedures cause inflammatory processes such as generalized redness, acne, rosacea, and increased susceptibility to contact and irritant dermatitis to wound dressings and other forms of inflammation during the healing process. Such procedures may result in adverse events such a pigment alteration (hypopigmentation and hyperpigmentation), acne lesions, and/or scarring. In some cases, the scarring and pigmentation may be due to the procedure and, in other cases, it may be due to the acne resulting from the procedure. Acne in particular leads to red macules that persist for months often followed by hyperpigmented brown macules. Acne may occur from the procedure itself, or may be caused by the wound care dressings and/or skincare products used after the procedure.

### Skincare Composition

The methods of embodiments described herein may comprise treating the acneic or acne-prone skin with a skincare composition including: (a) an amphoteric surfactant, preferably an ampholyte, and/or a zwitterionic surfactant having an isoelectric point of pH 7 or below; (b) a non-ionic surfactant, preferably a polyglucoside; (c) colloidal silver; (d) an acid to complex with the colloidal silver, where the molar ratio of the acid to the colloidal silver (c) is at least 1:1 (or greater); and (e) one or more hydroxyacid(s). In some embodiments, the non-ionic surfactant is a polyglucoside. In some embodiments, the amphoteric surfactant is an ampholyte

The skincare compositions of embodiments herein may also be used on skin presenting with inflammatory dermatoses, rosacea, or folliculitis/pseudofolliculitis barbae. In some embodiments, the skincare compositions may also be used in conjunction with redness, acne, inflammation, rosacea, dermatitis, scarring, and pigmentation associated with post-laser or post-skin resurfacing procedure.

In some embodiments, the skincare composition may be made by: (i) adding the surfactants (a) and (b) to water (aqueous phase); (ii) optionally, adding a pearlizing agent with heat to the aqueous phase; (iii) adding colloidal silver, mixing until a homogenous dispersion is formed; (iv) adding an acid to complex with the colloidal silver, where the molar ratio of the acid (c) to the colloidal silver (d) is at least 1:1 (or greater); and, thereafter, (v) adding one or more hydroxyacid(s).

In some embodiments, a skincare composition may include: (a) an amphoteric surfactant, preferably an ampholyte, and/or a zwitterionic surfactant having an isoelectric point of pH 7 or below; (b) a non-ionic surfactant; (c) a complex formed by colloidal silver and an acid; and (d) one or more hydroxyacid(s). In some embodiments, the non-ionic surfactant is a polyglucoside. In some embodiments, the amphoteric surfactant is an ampholyte. In some embodiments, a skincare composition may include

As a first component, the skincare composition of embodiments described herein contains an amphoteric surfactant, preferably an ampholyte, and/or a zwitterionic surfactant having an isoelectric point of pH 7 or below. As will be understood by the person having ordinary skill in the chemical arts, amphoteric surfactants can exist with anionic (−), cationic (+), or zwitterionic (− and +) charges, depending on the pH. Ampholytes are surfactants with a fully-quaternized nitrogen and, consequently, cannot lose their positive charges.

Alkylamido alkylamines are a preferred class of ampholytes used in the skincare compositions used in accordance with the methods of embodiments described herein.

A particularly preferred ampholyte is Sodium Cocoamphoacetate which generally to the formula:

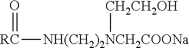

and is commercially available from a number of raw materials suppliers, including Rhodia Inc. (Cranbury, N.J.) under the tradename Miranol Ultra C 32.

Betaines, which for purposes of the present application include sultaines, are quaternized alkyl or substituted alkyl derivatives of N,N-dimethyl glycine, and are a preferred zwitterinoic surfactant used in formulating the skincare formulations used in accordance with the methods of embodiments described herein. Betaines have the structure:

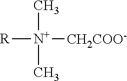

in which the nitrogen atom always carries a positive charge, regardless of pH.

Two preferred zwitterionic surfactants are Cocamidopropyl Hydroxysultaine and Cocamidopropyl Betaine.

Cocamidopropyl Hydroxysultaine conforms to the structure:

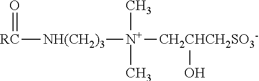

and is commercially available from a number of raw materials suppliers, including under the tradename Mirataine CBS from Rhodia, Inc. (Cranbury, N.J.).

Cocamidopropyl Betaine conforms generally to the structure:

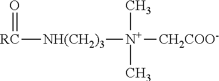

and is commercially available from, among other raw materials suppliers, Evonik Industries AG (Essen, Germany) under the tradename Tego Betain.

In some embodiments, the skincare formulation of embodiments described herein contains, as second component, a non-ionic surfactant, such as an ether of a fatty alcohol or an alkyl polyglucoside (APG). In some embodiments, the non-ionic surfactant is an APG. In some embodiments, the APG is Lauryl Glucoside, the product of condensation of lauryl alcohol with glucose. Lauryl Glucoside is commercially-available from a number of sources, including BASF Care Creations (Florham Park, N.J.) under the tradename Plantaren1200 N UP.

In certain embodiments, the skincare formulation of embodiments described herein contains, as a third required ingredient, colloidal silver—a suspension of silver particles in water prepared by electrolysis or by reduction of soluble silver salts. Colloidal silver is available from a number of suppliers of raw materials used in the manufacture of personal care products, including under the tradename Nanomate-Pure Nano Silver from Solco Nanoadvance Co., Ltd. (Changwon, South Korea), under the tradename OriStar CS from Orient Stars LLC (Torrance, Calif.), and under the tradename Permachem Ag+ from Permachem Asia Ltd. (Tokyo, Japan). Colloidal silver is also supplied by Solco Nanoadvance Co., Ltd. (Changwon, South Korea) as Sarpu Silver, and by Natural Immunogenics Corp. (Pompano Beach, Fla.) as Silver Hydrosol.

In some embodiments, the formulation also contains an acid that complexes with the colloidal silver to form a water-soluble silver salt. The molar ratio of the acid to the colloidal silver (c) may be at least (or greater than) 1:1.

Preferably, the acid that complexes with the colloidal silver is citric acid, an organic acid that conforms to the structure:

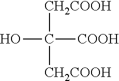

The resulting silver salt—silver citrate—in turn, will dissociate into ionic silver. Citric acid may be added in any form, for example, as grapefruit extract.

In another embodiment, the skincare formulation of embodiments described herein also includes a water-soluble silver salt of citric acid. This compound—INCI Name Citric Acid (and) Silver Citrate—corresponds to the structure:

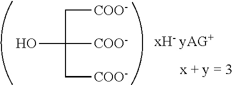

and is available from Pure Bioscience (El Cajon, Calif.) under the tradename SILVERION™ 2400. Preferably, SILVERION™ 2400 is present in the skincare formulation at a concentration of about 0.1 to about 0.3%.

The skincare formulation of some embodiments may also include at least one hydroxy acid, preferably salicylic acid. Salicylic acid is a phenolic, hydrophobic compound that induces exfoliation, and conforms to the structure:

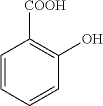

In certain embodiments, the skincare formulation may also include an alpha hydroxy acid. In some embodiments, the alpha hydroxy acids are glycolic acid [HOCH₂COOH] and lauric acid [CH₃(CH₂)₁₀COOH].

Importantly, in some embodiments, anionic surfactants are not used in making the skincare formulations used in the methods of embodiments described herein.

In some embodiments, the skincare compositions of embodiments herein may further include a preservative that is stable at pH 4 or less. Non-limiting examples of preservatives meeting this criterion include (i) Phenoxyethanol (and) Hexylene Glycol (and) Caprylyl Glycol sold under the tradename Diocide by Centerchem Inc. (Norwalk, Conn.) and (ii) Phenoxyethanol (and) Methylparaben (and) Ethylparaben (and) Butylparaben (and) Propylparaben (and) Isobutylparaben sold under the tradename Phenonip. In some embodiments, the skincare composition further includes a pearlizing or conditioning agent. Preferably, the solution is low-viscosity, typically less than <200 centipoise.

In some embodiments, the skincare composition further includes an acne treatment ingredient, such as benzoyl peroxide, clindamycin, erythromycin, polyhydroxy acid, willowbark extract, salicylic acid, resorcinol, resorcinol monoacetate, niacinamide, sulfur, beta carotene, vitamin A or derivatives thereof, retinol, tretinoin, adapalene, tazarotene, retinyl palmitate, retinyl linoleate, other ingredients that bind Toll-like 2 receptors, or a combination thereof. In some embodiments, the acne treatment ingredient is in nanoparticle form. In some embodiments, the benzoyl peroxide is in an amount of about 0.5%, about 1%, about 1.5%, about 2%, about 2.5%, about 3%, about 3.5%, about 4%, about 4.5%, about 5%, about 6%, about 7%, about 8%, about 9%, about 10%, or in a range between any two of the foregoing values. In some embodiments, the resorcinol may be in an amount of about 0.5%, about 1%, about 1.5%, about 2%, about 2.5%, about 3%, or in a range between any two of the foregoing values. In some embodiments, the salicylic acid may be in an amount of about 0.5%, about 0.75%, about 1%, about 1.5%, about 2%, or in a range between any two of the foregoing values. In some embodiments, the sulfur may be in an amount of about 2.5%, about 3%, about 3.5%, about 4%, about 4.5%, about 5%, about 6%, about 7%, about 8%, about 9%, about 10%, or in a range between any two of the foregoing values. In some embodiments, the acne treatment ingredient may be resorcinol monoacetate and sulfur, wherein the sulfur is in an amount of about 3% to about 8% and resorcinol monoacetate in an amount of about 3%. In some embodiments, the skincare composition may be formulated as a cleanser, a toner, an ointment, a gel, a cream, a lotion, a solution, or the like. For example, the skincare composition may be a cleansing gel, a liquid toner, a cream cleanser, a cream spot treatment, a spot treatment gel, or the like. In certain embodiments, the skincare composition may be formulated as part of a brush, spray, sponge, wipe or pad. For example, the skincare composition may be a cleansing wipe, a cleansing pad, a toner wipe, a toner spray, or a toner pad.

### Textile Containing Therapeutic-Effective Amount of Silver

The methods of embodiments described herein include the step of contacting acneic skin, acne-prone skin, skin exhibiting an inflammatory dermatosis, folliculitis, or pseudofolliculitis barbae with a textile containing a therapeutically-effective amount of silver.

As used in describing the textile containing/coated with a therapeutically-effective amount of silver, or fibers/strands used to manufacture the textile, the terms “coated” or “coating” are to be understood to describe a process by which the textile, fiber, or strand is dipped, immersed, or otherwise covered in silver, or its salts, or polymers containing the same. In some embodiments, the term “coated” is to be understood to mean that the silver (alone or in combination with one or more other metals, preferably zinc/or copper, or in combination with other nanoparticles, such as benzoyl peroxide or salicylic acid), is deposited, bound or otherwise affixed to the textile or fibers/strands from which the textile is formed. Coating may include injection, spray, dripping, or any other means that achieves uniform application onto the textiles. For example, a layer of 99.9% pure metallic silver may be permanently bonded onto the textile to coat 100% of the strand permanently. When the silver is oxidized with exposure to moisture, body heat, or air, positively charged silver ions are released resulting in a 4-log reduction of the negatively charged bacteria within one hour. This technology may or may not release metallic silver and maintains the flexibility of the fabric. In some embodiments, the silver textile produced from this method releases silver ions in the form of Ag, Ag⁺, and/or Ag⁺⁺.

In some embodiments, the textile and/or fiber from which the textile is made, may be coated with two layers of treatment materials—for example, a first layer comprising silver and a second layer (over the first layer) comprising zinc and/or copper.

Silver nanoparticles may be coated onto the surface of fibers, filaments and/or fabric by techniques well-known in the art, including those described in the scientific papers by S. Perera, et al., H. Xu, et al., and B. Mahltig, et al., and U.S. Pat. No. 8,183,167, each discussed in the Background of Invention.

Alternatively, silver may be incorporated into a polymer material that is applied to (i.e., coated) onto the fibers, filaments and/or fabric. In such an embodiment, silver at or proximate to the surface of the fiber or filament is available to provide antimicrobial action (i.e., killing P. acnes). The above method of creating antimicrobial textiles with silver ions is illustrated in U.S. Pat. No. 7,754,625.

In certain embodiments, a colloidal silver solution is sprayed onto a textile, which, in one preferred embodiment, is a pillowcase, in a cationic solution comprising (i) at least one quaternary compound, preferably selected from (a) diamido amine, (b) imidazolinium and (c) triethylamine (TEA), and (ii) silver salt or colloidal silver. Diamido amine quaternaries, imidazolinium quaternaries, and TEA suitable for use in spray formulations containing silver salt or colloidal silver are available from a number of chemical suppliers, including Abitec Corp. (Columbus, Ohio).

Non-limiting examples of suitable imidazolinium compounds are tallow imidazolinium quaternary, ditallow imidazolinium quaternary, dialkyl imidazolinium quaternary, imidazolinium methosulfate, dioleyl imidazolinium methosulfate, alkylamidoethyl alklyl imidazolinium methyl methosulfate, dicoco imidazolinium quaternary, dioleic imidazolinium quaternary, methyl-1-tallow amidoethyl-2-tallow imidazolinium methosulfate and methyl-1-oleyl amidoethyl-2-oleyl imidazolinium methosulfate.

In some embodiments, the textile is treated with silver, silver salts, silver nanoparticles, or a combination thereof in a batch process. For example, the textile along with the treatment materials (e.g. silver) may be loaded into an enclosed machine and mixed in the machine using specific rotation cycles. In some embodiments, the enclosed machine may be a front loading washing machine. In some embodiments, the cylinder of the machine is rotated clockwise for a specific cylinder rotation time and cylinder speed, followed by counterclockwise rotation for a specific time and cylinder speed. In some embodiments, the cylinder rotation time may be about 1 second to about 600 seconds in the clockwise direction, and, in the counterclockwise direction, the cylinder rotation time may be about 1 second to about 600 seconds. In some embodiments, the cylinder speed is about 1 rpm to about 100 rpm. Rotation speed and times may be optimized for various product configuration and for the amount of product that is loaded into the machine. To achieve uniform application and distribution of the treatment material being applied, speeds may be held constant or may be varied during the same rotation cycle or from clockwise to counterclockwise rotation. The treatment materials may be added to the machine while the textiles are in motion inside the machine. The treatment materials may be added into the machine by any applicable method, including, without limitations, by injection, spray, dripping, or any other means that achieves uniform application onto the textiles as they are moving or mixing inside the machine. In some embodiments, the treatment is sprayed into the machine at a rate and droplet size that achieves consistent application to the products or materials. Droplet size can vary from fine mists of sub-micron size to micron size, to millimeter size droplets.

Multiple treatment materials (e.g. silver and zinc) may be applied in the process by applying a mixture of the treatment materials in one treatment, or each treatment material can be applied in separate treatment cycles. For example, in some embodiments, silver may be combined with zinc prior to application. In some embodiments, silver may be applied to the textiles, followed by a separate application of zinc to the textile. In some embodiments, stabilizers may be used as needed to create stable mixtures prior to application. In some embodiments, additives may be used to achieve desired efficacy and longevity of the treated products. In some embodiments, treatment materials may be applied as individual components in successive application cycles to avoid chemical reactions, coagulation or other undesirable interactions that may occur if components are mixed in liquid form. In some embodiments, application of the treatment materials may take 1, 2, 3, 4, or more treatment cycles. Using additional treatment cycles may avoid the mixing of incompatible treatment materials. Once a treatment material is applied on the textile, another layer of a second treatment material on top of the existing layer may be used to achieve coatings on the textile that cannot be achieved by premixing the treatment materials. During application, in some embodiments, the treated textile may be dried between successive applications. In some embodiments, the treatment material can be added in layers (separate spraying, injection, dripping, etc. cycles) onto a treated textile that has not been dried.

Utilizing the above batch process technique yields application of treatment materials having commercially acceptable consistency while generating significantly less wastewater than other methods of application. The technique also provides excellent traceability of treated products, and provides a clean and sanitary method of application. Machines of various sizes and configurations may be used to achieve treatment of small quantities of products (e.g. textiles) as low as one pound as well as large quantities of products up to and exceeding 800 pounds of products.

In some embodiments, the textile may be topically treated with the treatment materials (e.g. silver) by saturating the product in a treatment solution followed by squeezing using a device that achieves uniform squeeze roll pressure.

In some embodiments, silver may be added to the textile in any amount that is therapeutically effective. In some embodiments, the silver is in an amount of less than about 10 ppm, about 10 to about 100 ppm, about 10 to about 90 ppm, about 10 to about 80 ppm, about 10 to about 70 ppm, about 10 to about 60 ppm, about 10 to about 50 ppm, about 10 to about 40 ppm, about 10 to about 30 ppm, about 10 to about 20 ppm, or a range therein. In some embodiments, the silver is in an amount of about 10 ppm, 20 ppm, 30 ppm, 40 ppm, 50 ppm, 60 ppm, 70 ppm, 80 ppm, 90 ppm, 100 ppm, greater than 100 ppm, or a range between any two of these values.

In some embodiments, the treatment materials used in coating the textile or fibers/strands are nanoparticles, preferably having an average particle size of about 1 nm to about 500 nm. In some embodiments, the textile is coated with a water-soluble salt of silver. In some embodiments, the treatment materials may include silver, copper, nickel, titanium, cobalt, gold, iron, chromium, platinum, plutonium, mercury, nickel, magnesium, cadmium, gadolinium, lithium, tungsten, zinc, benzoyl peroxide, clindamycin, erythromycin, polyhydroxy acid, willowbark extract, salicylic acid, resorcinol, resorcinol monoacetate, sulfur, beta carotene, vitamin A or derivatives thereof, retinol, tretinoin, adapalene, tazarotene, retinyl palmitate, retinyl linoleate, other ingredients that bind Toll-like 2 receptors, other retinoids, poly hydroxyacid, tannic acid, hydroxybenzoic acid, juniperic acid, tartaric acid, glycolic acid, lactic acid, citric acid, lauric acid, tyrosinase inhibitors, PAR-2 blockers, lignin peroxidase, or a combination thereof. In some embodiments, the silver coated onto the textile is colloidal silver. In particular embodiments, the textile includes silver and zinc. In some embodiments, the textile includes silver and salicylic acid. In some embodiments, the textile includes silver and benzoyl peroxide. In some embodiments, the textile includes silver and retinoid selected from tretinoin, adapalene, retinol, tazarotene, or a combination thereof. In some embodiments, the textile comprises silver and sulfur. In some embodiments, the textile comprises silver, benzoyl peroxide and a retinoid. In some embodiments, the retinoid may be selected from tretinoin, adapalene, retinol, tazarotene or a combination thereof. In some embodiments, the textile comprises silver and an acne treatment ingredient. The acne treatment ingredient may be selected from benzoyl peroxide, clindamycin, erythromycin, polyhydroxy acid, willowbark extract, salicylic acid, resorcinol, resorcinol monoacetate, niacinamide, sulfur, beta carotene, vitamin A or derivatives thereof, retinol, tretinoin, adapalene, tazarotene, retinyl palmitate, retinyl linoleate, other ingredients that bind Toll-like 2 receptors, other retinoids, poly hydroxyacid, tannic acid, hydroxybenzoic acid, juniperic acid, tartaric acid, glycolic acid, lactic acid, citric acid, lauric acid, tyrosinase inhibitor, PAR-2 blocker, lignin peroxidase, or a combination thereof. In some embodiments, the textile may further include a depigmenting ingredient. The depigmenting treatment ingredient may be selected from tyrosinase inhibitor, PAR-2 blocker, lignin peroxidase or a combination thereof. Examples of a tyrosinase inhibitors that may be used in embodiments herein include hydroquinone, kojic acid, arbutin, mulberry extract, and ascorbic acid. Example of a PAR-2 blocker that may be used in embodiments herein include soy and niacinamide. In some embodiments, the acne treatment ingredient, depigmentating treatment ingredient, and/or colloidal silver is on the same fiber of the textile. In some embodiments, the acne treatment ingredient, depigmenting treatment ingredient, and/or colloidal silver is not on the same fiber of the textile. In some ingredients, the acne treatment ingredient is in the form of nanoparticles, microparticles, microspheres, micron sized particles, microencapsulated materials, matrices, coated particles, smart particles responsive to environmental stimuli, UV curable particles, low temperature melt particles, or a combination thereof. In some embodiments, the depigmenting treatment ingredient is in the form of nanoparticles, microparticles, microspheres, micron sized particles, microencapsulated materials, matrices, coated particles, smart particles responsive to environmental stimuli, UV curable particles, low temperature melt particles, or a combination thereof.

In certain embodiments the textile is machine washable. In some embodiments, the textile is reusable. In some embodiments, the textile is disposable.

While each strand of the textile may be coated with silver (alone or in combination with one or more other metals, preferably zinc/or copper, or other treatment materials, such as one or more acne treatment ingredients), in one preferred embodiment, every fifth strand/fiber used in fabricating the textile is coated with silver (alone or with one or more other metals, preferably zinc/or copper, or with other treatment materials, such as one or more acne treatment ingredients). It will be appreciated that textiles made from a fabric in which every second, third, fourth, etc. strand are also included within the scope of the invention.

In some embodiments, the silver textile is further coated with an acne treatment ingredient such as benzoyl peroxide, clindamycin, erythromycin, polyhydroxy acid, willowbark extract, salicylic acid, resorcinol, resorcinol monoacetate, niacinamide, sulfur, beta carotene, vitamin A or derivatives thereof, retinol, tretinoin, adapalene, tazarotene, retinyl palmitate, retinyl linoleate, other ingredients that bind Toll-like 2 receptors, other retinoids, poly hydroxyacid, tannic acid, hydroxybenzoic acid, juniperic acid, tartaric acid, glycolic acid, lactic acid, citric acid, lauric acid, or a combination thereof. In some embodiments, the acne treatment ingredient is in nanoparticle form. In some embodiments, the benzoyl peroxide is in an amount of about 0.5%, about 1%, about 1.5%, about 2%, about 2.5%, about 3%, about 3.5%, about 4%, about 4.5%, about 5%, about 6%, about 7%, about 8%, about 9%, about 10%, or in a range between any two of these values. In some embodiments, the resorcinol may be in an amount of about 0.5%, about 1%, about 1.5%, about 2%, about 2.5%, about 3%, or in a range between any two of the foregoing values. In some embodiments, the salicylic acid may be in an amount of about 0.5%, about 0.75%, about 1%, about 1.5%, about 2%, or in a range between any two of the foregoing values. In some embodiments, the sulfur may be in an amount of about 2.5%, about 3%, about 3.5%, about 4%, about 4.5%, about 5%, about 6%, about 7%, about 8%, about 9%, about 10%, or in a range between any two of the foregoing values. In some embodiments, the acne treatment ingredient may be resorcinol monoacetate and sulfur, wherein the sulfur is in an amount of about 3% to about 8% and resorcinol monoacetate in an amount of about 3%.

### Regimens and Kits

Some embodiments are directed to kits comprising a skincare composition of embodiments herein and a silver textile of embodiments herein. In some embodiments, a skincare kit for treating acne comprises a skincare composition comprising colloidal silver and a silver textile. In some embodiments, the skincare composition comprising colloidal silver may further include an amphoteric surfactant and/or a zwitterionic surfactant having an isoelectric point of pH 7 or below; a non-ionic surfactant; an acid to complex with the colloidal silver, where the molar ratio of the acid to the colloidal silver is at least 1:1 (or greater); and one or more hydroxyacid(s). In some embodiments, the skincare composition comprising colloidal silver is a cleanser, a toner, an ointment, a gel, a cream, a lotion, a solution, or a combination thereof. In some embodiments, the silver textile is a shirt, pillowcase, or pajamas coated with colloidal silver. In some embodiments, the silver textile is coated with silver nanoparticles. In some embodiments, the silver textile is further coated with an acne treatment ingredient. In some embodiments, the acne treatment ingredient is selected from benzoyl peroxide, clindamycin, erythromycin, polyhydroxy acid, willowbark extract, salicylic acid, resorcinol, resorcinol monoacetate, niacinamide, sulfur, beta carotene, vitamin A or derivatives thereof, retinol, tretinoin, adapalene, tazarotene, retinyl palmitate, retinyl linoleate, other ingredients that bind Toll-like 2 receptors, other retinoids, poly hydroxyacid, tannic acid, hydroxybenzoic acid, juniperic acid, tartaric acid, glycolic acid, lactic acid, citric acid, lauric acid, tyrosinase inhibitor, PAR-2 blocker, lignin peroxidase, or a combination thereof. In some embodiments, the textile may further include a depigmenting ingredient. The depigmenting treatment ingredient may be selected from tyrosinase inhibitor, PAR-2 blocker, lignin peroxidase or a combination thereof. Examples of a tyrosinase inhibitors that may be used in embodiments herein include hydroquinone, kojic acid, arbutin, mulberry extract, and ascorbic acid. Example of a PAR-2 blocker that may be used in embodiments herein include soy and niacinamide. In some embodiments, the kit further includes an acne treatment composition comprising an acne treatment ingredient. In some embodiments, the acne treatment ingredient in the acne treatment composition is benzoyl peroxide or a retinoid. In some embodiments, the retinoid may be selected from retinol, retinyl linoleate, retinyl palmitate, adapalene, tretinoin tazarotene, or a combination thereof. In some embodiments, the acne treatment composition may be a cream or a gel. In some embodiments, the kit comprises a cleanser comprising the skincare composition of embodiments herein and a silver textile of embodiments herein. In some embodiments, the hydroxy acid in the cleanser may be salicylic acid or willowbark extract. In some embodiments, the cleanser may be in the form of a cleansing wipe, a liquid, a foaming cleanser, a gel, or the like. In some embodiments, the kit comprises a toner comprising the skincare composition of embodiments herein. In some embodiments, the hydroxy acid in the toner is salicylic acid. In some embodiments, the kit comprises a cleanser comprising the skincare composition of embodiments herein, a toner comprising the skincare composition of embodiments herein, and a silver textile of embodiments herein. In some embodiments, the kit comprises a cleanser comprising the skincare composition of embodiments herein; an acne treatment composition comprising benzoyl peroxide; and a silver textile of embodiments herein. In some embodiments, the kit comprises a toner comprising the skincare composition of embodiments herein; an acne treatment composition comprising benzoyl peroxide; and a silver textile of embodiments herein. In some embodiments, the kit comprises a cleanser comprising the skincare composition of embodiments herein; a toner comprising the skincare composition of embodiments herein; an acne treatment composition comprising benzoyl peroxide; and a silver textile of embodiments herein.

Some embodiments are directed to an acne kit comprising a silver textile of embodiments herein, a cleanser comprising silver, and an acne treatment composition. In some embodiments, the acne treatment composition comprises benzoyl peroxide or a retinoid. In some embodiments the cleanser may be a liquid, a gel, a foam, a wipe, a pad, or the like. In some embodiments the cleanser may be a cleansing wipe comprising silver, wherein the cleanser may be sprayed with silver, or the fibers of the wipe are coated or otherwise treated with silver. In some embodiments, the acne kit further comprises a toner comprising silver. In some embodiments, the toner is a spray, liquid, gel, foam, a wipe, or the like. Some embodiments are directed to an acne kit comprising a silver textile of embodiments herein, a toner comprising silver, and an acne treatment composition. In some embodiments, the toner is a spray, liquid, gel, foam, a wipe, or the like.

In some embodiments, an acne kit for body acne may comprise a shirt comprising a silver textile of embodiments herein, an acne treatment composition, and a cleanser comprising a skincare composition of embodiments herein. In some embodiments, an acne kit for body acne may comprise a shirt comprising a silver textile of embodiments herein, an acne treatment composition, and a toner comprising a skincare composition of embodiments herein. In some embodiments, an acne kit for body acne may comprise a shirt comprising a silver textile of embodiments herein, an acne treatment composition, a cleanser comprising a skincare composition of embodiments herein, and a toner comprising a skincare composition of embodiments herein.

In some embodiments, an acne kit for facial acne may comprise a pillowcase and/or mask comprising a silver textile of embodiments herein, an acne treatment composition, and a cleanser comprising a skincare composition of embodiments herein. In some embodiments, an acne kit for facial acne may comprise a pillowcase and/or mask comprising a silver textile of embodiments herein, an acne treatment composition, and a toner comprising a skincare composition of embodiments herein. In some embodiments, an acne kit for facial acne may comprise a pillowcase and/or mask comprising a silver textile of embodiments herein, an acne treatment composition, a cleanser comprising a skincare composition of embodiments herein, and a toner comprising a skincare composition of embodiments herein.

Embodiments herein are also directed to a skincare regimen for treating acne, the regimen comprising: washing the affected area with a cleanser of embodiments herein, applying a toner of embodiments herein to the affected area; and contacting the affected area with a silver textile of embodiments herein for at least about one hour. In some embodiments, the skin care regimen for treating acne may further include applying an acne treatment composition comprising nanoparticles of an active to the affected area. In some embodiments, the acne treatment composition may be applied only to acne lesions (spot treatment) or may be applied to the entire affected area, such as the face, back or chest. Some embodiments herein are directed to a skin care regimen to treat acne, the regimen comprising a morning routine of applying a toner of embodiments herein to the affected area, and an evening routine of washing the affected area with a cleanser of embodiments herein, and contacting the affected area with a silver textile of embodiments herein for at least about one hour. In some embodiments, the evening routine may further include applying an acne treatment composition of embodiments herein. In some embodiments, the acne treatment composition may be applied to the lesions or to the entire affected area. In some embodiments, the affected area has mild to moderate acne, moderate acne, moderate to severe acne, or severe acne. Some embodiments are directed to a skin care regimen to treat acne, the regimen comprising a morning routine including washing the affected area with a cleanser of embodiments herein, and applying a toner of embodiments herein to the affected area; and an evening routine including washing the affected area with a cleanser of embodiments herein, applying a toner of embodiments herein, applying an acne treatment composition of embodiments herein, and contacting the affected area with a silver textile of embodiments herein for at least one hour.

In some embodiments, a post-laser and/or post-skin resurfacing procedure healing kit may comprise a silver textile; a skincare composition in the form of a cleanser; a skincare composition in the form of a toner, an occlusive healing ointment comprising silver; and, optionally, a sunscreen. In some embodiments, the sunscreen comprises silver and zinc. In some embodiments, the sunscreen comprises a physical block sunscreen, a chemical absorber sunscreen, or a combination thereof. In some embodiments, a post-laser and/or post-skin resurfacing procedure skincare regimen on an affected area of skin, the regimen comprising a morning routine of cleansing the affected area with cleanser of embodiments herein; applying toner of embodiments herein to the affected area; applying an occlusive healing ointment comprising silver; and applying an SPF; and an evening/bedtime routine of cleansing the affected area with cleanser of embodiments herein; applying toner of embodiments herein to the affected area; applying an occlusive healing ointment comprising silver; and contacting the affected area with a silver textile for at least one hour.

Table 1 describes several exemplary regimens of embodiments herein. References to cleanser, toner, ointment, an acne treatment composition, and silver textile are references to those items described in the embodiments herein as well as those otherwise commercially available. In some embodiments, the regimens of embodiments herein may further include applying an SPF comprising silver, zinc oxide, or a combination thereof. If both face and body acne regimes are desired, they may be used in combination.

TABLE 1

| Regimen | AM Regimen | PM Regimen |
|---|---|---|
| Face Regimen 1 | 1. Apply Toner | 1. Wash face with cleanser |
| (to prevent acne) | | 2. Contact with silver textile |
| | | for at least one hour |
| Face Regimen 2 | 1. Apply Toner | 1. Wash face with cleanser |
| (Mild Acne) | | 2. Apply an acne treatment |
| | | composition to lesions only |
| | | 3. Contact with silver textile |
| | | for at least one hour |
| Face Regimen 3 | 1. Apply Toner | 1. Wash face with cleanser |
| (Mild to | | 2. Apply an acne treatment |
| Moderate Acne) | | composition to acne lesion or |
| | | entire face |
| | | 3. Contact with silver textile |
| | | for at least one hour |
| Face Regimen 4 | 1. Wash face with cleanser | 1. Wash face with cleanser |
| (Moderate Acne) | 2. Apply Toner | 2. Apply toner |
| | | 3. Apply an acne treatment |
| | | composition to entire face |
| | | 4. Contact with silver textile |
| | | for at least one hour |
| Face Regimen 5 | 1. Apply Toner | 1. Wash face with cleanser |
| (Moderate Acne) | | 2. Apply an acne treatment |
| | | composition to acne lesions |
| | | (spot treatment) or entire face |
| | | 3. Apply toner |
| | | 4. Contact silver textile for at |
| | | least one hour |
| Face Regimen 6 | 1. Apply Toner | 1. Wash face with cleanser |
| (Moderate to | 2. Apply spot treatment to | 2. Apply an acne treatment |
| Severe Acne) | lesions | composition to lesion or entire |
| | | face |
| | | 3. Contact with silver textile |
| | | for at least one hour |
| Face Regimen 7 | 1. Wash face with cleanser | 1. Wash face with cleanser |
| (Severe Acne) | 2. Apply toner | 2. Apply toner |
| | 3. Apply spot treatment | 3. Apply an acne treatment |
| | | composition to entire face |
| | | 4. Contact with silver textile |
| | | for at least one hour |
| Body Regimen 1 | 1. Wipe affected area (back or | 1. Wipe affected area (back or |
| (to prevent | chest) with cleansing wipe | chest) with cleansing wipe |
| acne) | 2. Spray toner on affected area | 2. Wear silver textile shirt to |
| | | sleep for at least one hour |
| Body Regimen 2 | 1. Wipe affected area (back or | 1. Wipe affected area (back or |
| (Mild Acne) | chest) with cleansing wipe | chest) with cleansing wipe |
| | 2. Spray toner on affected area | 2. Spray toner on affected area |
| | | 3. Wear silver textile shirt to |
| | | sleep for at least one hour |
| Body Regimen 3 | 1. Wipe affected area (back or | 1. Wipe affected area (back or |
| (Moderate Acne) | chest) with cleansing wipe | chest) with cleansing wipe |
| | 2. Spray toner on affected area | 2. Spray toner on affected area |
| | | 3. Apply an acne treatment |
| | | composition to lesion |
| | | 4. Wear silver textile shirt to |
| | | sleep for at least one hour |
| Body Regimen 4 | 1. Wipe affected area (back or | 1. Wipe affected area (back or |
| (Severe Acne) | chest) with cleansing wipe | chest) with cleansing wipe |
| | 2. Spray toner on affected area | 2. Spray toner on affected area |
| | 3. Apply an acne treatment | 3. Apply an acne treatment |
| | composition to entire affected | composition to lesion (spot |
| | area | treatment) |
| | | 4. Wear silver textile shirt to |
| | | sleep for at least one hour |
| Post laser or | 1. Wash area that has had the | 1. Wash area that has had the |
| skin resurfacing | procedure (face or body) with | procedure (face or body) with |
| procedure | cleanser (w/no salicylic acid) | cleanser (w/no salicylic acid) |
| regimen 1 (to | 2. Spray toner on affected area | 2. Spray toner on affected area |
| prevent acne) | (no salicylic acid) | (no salicylic acid) |
| | | 3. Use silver textile |
| | | (pillowcase or shirt) at |
| | | bedtime/during sleep for at |
| | | least one hour |
| Post laser or | 1. Gently cleanse area with | 1. Gently cleanse area with |
| skin resurfacing | cleanser | cleanser |
| Procedure | 2. Apply toner on area | 2. Apply toner on area |
| regimen 2 (to | 3. Apply healing ointment (for | 3. Apply healing ointment (for |
| prevent acne and | 7 days, omit after 7 days) | 7 days, omit after 7 days) |
| post-inflammatory | 4. Apply SPF | 4. Use silver textile |
| pigmentation) | | (pillowcase or shirt) at |
| (Use for | | bedtime/during sleep for at |
| 14-21 days) | | least one hour |

The skincare compositions of embodiments herein may also be formulated in other ways to treat acne in particular areas, such as a shampoo for treatment of scalp acne and/or folliculitis. In some embodiments, a method of treating scalp acne and/or folliculitis includes applying a shampoo comprising silver to the area to be treated. In some embodiments, the silver may be colloidal silver. In some embodiments, the silver may be in the form of nanoparticles, microparticles, microspheres, micron sized particles, microencapsulated materials, matrices, coated particles, smart particles responsive to environmental stimuli, UV curable particles, low temperature melt particles, or a combination thereof.

Though the regimens and treatments are described with reference to acne herein, they will be understood by one skilled in the art to be similarly used for the treatment of acne, rosacea, other inflammatory lesions, post-inflammatory pigmentation, lesions after skin resurfacing modalities, or the like.

Embodiments of the invention illustrating the method and materials used herein may be further understood by reference to the following non-limiting examples.

### EXAMPLE 1

### Rinse-Off Cleanser

| Ingredient | INCI Name | Wt % |
|---|---|---|
| Phase A | | |
| Miranol Ultra C32 | Sodium Cocoamphotacetate | 26.0 |
| Mirataine CBS | Cocamidopropyl Hydroxysultaine | 4.0 |
| Plantaren 1200 | Lauryl Polyglucoside | 5.0 |
| Texapon Z95P | Sodium Lauryl Sulfate | 0.5 |
| Glycerine | Glycerine | 1.0 |
| Deionized Water | Water | Q.S. |
| Phase B | | |
| Jaguar C14S | Guar Hydroxypropyltrimonium | 0.5 |
| | Chloride | |
| Phase C | | |
| Alkamuls EGDS | Glycol Distearate | 1.5 |
| Phase D | | |
| SILVERION 2400 or | (Citric Acid and Silver | 0.3 |
| Colloidal Silver | Citrate) or | |
| | (Colloidal Silver) | |
| Phase E | | |
| Citric Acid (if | Citric Acid | Adjust to |
| using Colloidal | | pH 5.5-6 |
| Silver) | | |
| Phase F | | |
| Salicylic Acid | Salicylic Acid | 1.0 |
| Lauric Acid | Lauric Acid | 0.3 |
| Phase G | | |
| Diocide | Phenoxyethanol (and) Hexylene | 0.7 |
| | Glycol (and) Caprylyl Glycol | |

Formulation of Example 1 is made as follows:

(i) Into water, add Phase A and mix well until homogenous. Disperse Phase B at room temperature into Phase A.

(ii) Heat Phase A/B to 70-75° C. and subsequently add Phase C while homogenizing without aeration. Cool Phase A/B/C at 30-35° C. and Phase D mixing well.

(iii) Add Phase E, as needed, to adjust final pH to 5.5-6.0. Add Phase F to Phase A/B/C/D/E while mixing. Add Phase G to Phase A/B/C/D/E/F and mix well. Package.

### EXAMPLE 2

### Cleanser

| Ingredient (INCI Name) | Wt % |
|---|---|
| Water | Q.S. |
| Aloe Barbadensis Leaf Extract | 3.00-15.00% |
| Betaine | 2.00-16.00% |
| Allantoin | 0.30-2.00% |
| Potassium Hydroxide | 0.20-2.00% |
| Colloidal Silver | 0.10-2.00% |
| Glycolic Acid | 0.10-2.00% |
| Dimethicone | 0.15-1.00% |
| Lauryl Glucoside | 0.10-0.60% |
| Tetrasodium EDTA | 0.01-0.10% |
| Benzalkonium Chloride | 0.005-0.13% |
| Phenoxyethanol; Methylparaben; Ethylparaben; | 0.30-2.00% |
| Butylparaben; Propylparaben; Isobutylparaben | |

### EXAMPLE 3

### Cleansing Gel

| Ingredient (INCI Name) | Wt % |
|---|---|
| Water | Q.S. |
| Salicylic Acid | 0.05-5.00% |
| Aloe Barbadensis Leaf Extract | 0.50-8.00% |
| Coco-Betaine | 1.00-45.00% |
| Cocamidopropyl Betaine | 0.10-8.00% |
| Allantoin | 0.10-2.00% |
| Sodium Cocoyl Isethionate | 1.00-45.00% |
| Acrylates Crosspolymer-4 | 0.30-9.00% |
| Colloidal Silver | 0.05-2.00% |
| Zinc PCA | 0.02-2.00% |
| PEG/PPG-4/12 Dimethicone | 0.05-4.00% |
| Lauryl Glucoside | 0.05-5.00% |
| Tetrasodium EDTA | 0.01-0.20% |
| Sodium Benzoate | 0.05-0.50% |
| Imidazolidinyl Urea | 0.05-0.50% |
| Potassium Sorbate | 0.05-0.50% |
| Citric Acid | 0.01-1.00% |
| Sodium Hydroxide | 0.02-1.00% |

### EXAMPLE 4

### Leave on Toner

| Ingredient | INCI | Wt % |
|---|---|---|
| Phase A | | |
| Miranol Ultra C32 | Sodium Cocoamphotacetate | 1.0 |
| Plantaren 1200 | Lauryl Polyglucose | 0.5 |
| Deionized Water | Water | Q.S. |
| Phase B | | |
| Jaguar C14S | Guar Hydroxypropyltrimonium | 0.1 |
| | Chloride | |
| Phase C | | |
| Aloe Vera (10X) | Aloe Barbadensis Leaf Extract | 0.1 |
| Phase D | | |
| SILVERION 2400 or | (Citric Acid and Silver | 0.1 |
| Colloidal Silver | Citrate) or | |
| | (Colloidal Silver) | |
| Phase E | | |
| Citric Acid (if | Citric Acid | Adjust to |
| using Colloidal | | pH 5.5-6 |
| Silver) | | |
| Phase F | | |
| Salicylic Acid | Salicylic Acid | 0.5 |
| Phase G | | |
| Diocide | Phenoxyethanol (and) Hexylene | 0.7 |
| | Glycol (and) Caprylyl Glycol | |

Formulation of Example 4 is made as follows:

(i) Into water, add Phase A and mix well until homogenous. Disperse Phase B and then Phase C at room temperature into Phase A.

(ii) Add Phase D to Phase A/B/C and mix well. Add Phase E, as needed, to adjust pH to 5.5-6.0.

(iii) Add Phase F to Phase A/B/C/D/E while mixing.

(iv) Add Phase G to Phase A/B/C/D/E/F and mix until homogenous. Package.

### EXAMPLE 5

### Leave on Toner

| Ingredient (INCI Name) | Wt % |
|---|---|
| Water | Q.S. |
| Salicylic Acid | 0.05-5.00% |
| Aloe Barbadensis Leaf Extract | 0.50-8.00% |
| Propanediol | 5.00-50.00% |
| Cocamidopropyl Betaine | 0.10-8.00% |
| Allantoin | 0.10-2.00% |
| Tocopheryl Acetate | 0.05-2.00% |
| Glycerin | 0.10-5.00% |
| Colloidal Silver | 0.05-5.00% |
| Citrus Grandis (Grapefruit) Fruit | 0.10-3.00% |
| Extract (includes citric acid) | |
| PEG/PPG-4/12 Dimethicone | 0.05-4.00% |
| Lauryl Glucoside | 0.05-5.00% |
| Tetrasodium EDTA | 0.01-0.20% |
| Sodium Benzoate | 0.05-0.50% |
| Diazolidinyl Urea | 0.05-0.50% |
| Potassium Sorbate | 0.05-0.50% |
| Sodium Hydroxide | 0.02-1.00% |

### EXAMPLE 6

### Acne Treatment Regimen Clinical Study

Forty patients having facial acne are enrolled in an eight-week clinical study after giving informed consent. In order to qualify for participation in the study, patients are clinically determined to have mild to moderate acne at baseline, as measured by the following criteria: no cysts, ≧8 inflammatory lesions (papules/pustules) and ≧4 non-inflammatory lesions (black heads/white heads) on each side of the face.

Patients are randomly divided into four groups and are assigned to follow one of four treatment groups, each of which is assigned to follow a different treatment regimen—designated Treatment A, Treatment B, Treatment C and Treatment D.

| | Treat- | Treat- | Treat- | Treat- |
|---|---|---|---|---|
| Product | ment A | ment B | ment C | ment D |
| Pillowcase in accordance | X | | X | |
| with invention (fabric | | | | |
| treated with | | | | |
| therapeutically-effective | | | | |
| amount of silver) | | | | |
| Cotton Pillowcase (control) | | X | | X |
| Cleanser in accordance | | | X | X |
| with invention (silver + | | | | |
| salicylic acid) | | | | |
| Cera Ve cleanser (control) | X | X | | |

Prior to commencement of the study, subjects complete a one-week washout using the Cera Ve cleanser, a sunscreen and a moisturizer. On Day 0 of the study, each participant receives a cleanser (which for Treatments C and D is a skincare formulation of embodiments described herein) and a pillowcase (which for Treatments A and C is a pillow case whose fibers are treated/coated with a therapeutically-effective amount of silver of embodiments described herein). Additionally, at the commencement of the study, participants receive the same sunscreen (for daily application), the same moisturizer (to be used as needed), and a daily diary in which to track product usage in accordance with the study directions (i.e., compliance) and adverse events. Subjects are blinded as to the type of pillowcase, but not the type of cleanser, to be used throughout the study. The investigators are blinded as to which treatment regimen the subjects are assigned.

In addition to the baseline assessment (Day 0), assessments are made at the end of weeks 1, 4 and 8 of the study. Assessments are a combination of (a) clinical observations by the investigator, including lesion counts, (b) clinical imaging (digital photographs and image analysis using the Canfield Visia CR system) and (c) subject-self assessments including self-administered questionnaires, which includes visibility of pores.

Statistical analysis indicates that Treatment C is the most effective at weeks 4 and 8 (as compared to the other treatments). Improvements in skin condition (reduction in acne lesion count) among participants following Treatment C are statistically significant. Treatments A and D show improvement as compared to Treatment B, with Treatment A being superior to Treatment D. Evaluation of the subject diaries show that subjects are more compliant with the use of the pillowcases than with the cleanser, which may account for greater lesion resolution in treatment groups using a pillowcase in accordance with the methods of the invention.

Subjects assigned to Treatment C anecdotally report that pimples clear faster (i.e., the cycle of papule to pustule to erosion to red macula to clear is observed to be more rapid). Participants following the Treatment C regimen show more lesion resolution at weeks 1 and 4 than the other groups suggesting faster clearing of lesions in this group, which would not be explained by increased compliance alone. Tolerance assessments demonstrate similar incidence and severity of irritation for all treatment groups. Treatment groups A and C exhibit less post inflammatory hyperpigmentation than the other groups.

Vaestro Image Analysis Toolkit (part of Canfield Visia CR system) counts the number of porphyrins, which correlates to amounts of P. acnes on the skin. Bacterial counts are observed to be significantly lower in Treatment C as compared to the other treatment groups. Treatment groups D and A, respectively, showed reduced porphyrin counts (versus baseline), but to a lesser extent.

### EXAMPLE 7

### Efficacy of an Acne Kit with and without a Silver Containing Pillowcase

The current study comprised of a full face model composed of 2 treatment groups comparing the efficacy and tolerability of an acne kit regimen with and without a silver containing textile pillowcase. The purpose of this study was to evaluate the use of a silver containing pillowcase in conjunction with an acne kit to see if addition of silver to the pillowcase textile sped the healing of acne lesions and reduced acne counts as compared to the acne kit alone.

Materials and Methods: The study was comprised of 40 subjects (acne treatment subjects) with greater than 4 inflammatory acne lesions. 20 subjects received Kit A and 20 subjects received Kit B. In addition, 6 subjects (post procedure subjects) with a history of acne who had undergone laser facial resurfacing or needling for acne scars were also recruited. 3 of these post procedure subjects received Kit A and 3 subjects received Kit B. Kit A contained: (1) Percale Cotton Pillowcase; (2) 2.5% BP Spot treatment; (3) 2% SA/0.5% Colloidal Silver Cleanser; (4) 0.5% SA/1% Colloidal Silver Toner. Kit B contained: (1) Silver Containing Pillowcase; (2) 2.5% BP Spot treatment; (3) 2% SA/0.5% Colloidal Silver Cleanser; and (4) 0.5% SA/1% Colloidal Silver Toner.

Subjects were instructed to use the cleanser twice a day followed by the toner. The BP spot treatment was applied only to acne lesions at night. Subjects were instructed to apply the toner followed by the BP spot treatment immediately prior to sleeping on the study pillowcase so that the skin would be moist from the toner at textile contact.

Subjects were seen at Day 0, Day 30 (+/−5 days), Day 60 (+/−10 days), Day 90 (+/−15 days). The day of the resurfacing treatment was Day 0 for the subjects that were treated post resurfacing. Canfield photographs with porphyrin counts were taken at each visit. Subject assessments and blinded investigator assessments were performed at each visit. The blinded investigator performed lesion counts at each visit. The patients kept a subject diary for the first 30 days of the study.

The standard percale pillowcase in kit A was 100% cotton. The silver textile pillowcase in kit B was a standard size percale cotton pillowcase treated in a washing machine with a silver containing solution. Pillowcases were placed in a zip-lock bag labelled as Kit A or Kit B and the subject number. There was no color difference in the pillowcases and all efforts were made to control the blind of the study. The spot BP treatment was applied only to inflammatory acne lesions (papules and pustules).

Results: Acne scale—Statistical analysis indicated that Kit B was more effective as an acne treatment than Kit A at Day 30, Day 60 and Day 90. This was true for both the acne subjects and the post procedure subjects. Acne subjects using Kit B demonstrated more rapid healing of lesions (time from papule lesion to clear), fewer pustular lesions, and shorter duration of red macules as compared to Kit A acne subjects. Subjects using Kit B had fewer inflammatory and non-inflammatory acne lesions at each visit compared to subjects using Kit A and less resulting hyperpigmentation. The 3 post procedure subjects that used Kit B experienced no acne lesions and no hyperpigmentation after the procedure while 2 of the 3 post procedure subjects using Kit A experienced inflammatory (papules and pustules) acne lesions and 1 experienced diffuse hyperpigmentation in the treated area (the other had pigmentation limited to the site of the acne lesion).

Evaluation of the subject diaries showed an average healing time from papule to pink macule to be 3 days faster in the Kit B group as compared to 5 days in the Kit A group. Average healing time from “papule” to “clear” was an average of 10 days in the Kit B group compared to 17 days in the Kit A group.

The Kit B group had an inflammatory lesion count that was less than the Kit A group inflammatory lesions at day 30, 60 and 90 as compared to the Kit A group. Porphyrin levels as seen on the canfield CA camera system showed a significant difference at Day 30, 60 and 90 when the Kit A and Kit B group were compared. Average porphyrin levels at Day 30 for Kit A group were 4% and Kit B group was 40%. In the Canfield camera a lower percent number on the porphyrin count is associated with a higher P. acnes bacteria count.

Weighing of the BP spot treatment at each visit showed less utilization of the BP spot treatment in the Kit B group as compared to Kit A. (The Kit B spot treatment tubes weighed more compared to Kit A). Only the 40 “acne lesion” subjects tube weights were calculated—the “post procedure” subject tube weights were not included in this calculation.

This study clearly showed that the lesion progression was much faster in the Kit B group compared to the Kit A group. This likely occurs because the silver ions increased skin healing speed. This effect was not noted in previous trials that looked at SA and silver pillowcases and their effect on acne. It is likely that the addition of the BP spot treatment dramatically and unexpectedly increased the release of silver ions to an extent that sped skin healing. Additionally, subjects that received Kit B reported that the skin healed faster than those who used Kit A.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in embodiments described herein, including all features which would be treated as equivalents thereof by those skilled in the art to which the invention pertains.

1. A skincare composition comprising:
an amphoteric surfactant and/or a zwitterionic surfactant having an isoelectric point of pH 7 or below;
a non-ionic surfactant;
colloidal silver;
an acid to complex with the colloidal silver, where the molar ratio of the acid to the colloidal silver is at least 1:1 (or greater); and
one or more hydroxyacid(s).

2. The skincare composition of claim 1 wherein the amphoteric surfactant is an ampholyte.

3. The skincare composition of claim 2 wherein the ampholyte is sodium cocoamphoacetate.

4. The skincare composition of any of claims 1, wherein the zwiterrionic surfactant is a betaine.

5. The skincare composition of 4 wherein the zwiterrionic surfactant is cocamidopropyl hydroxysultaine or cocamidopropyl betaine.

6. The skincare composition of any of claim 1, wherein the non-ionic surfactant is an alkylpolyglucoside.

7. The skincare composition of claim 6, wherein the non-ionic surfactant is lauryl glucoside.

8. The skincare composition of any of claim 1, wherein the one or more hydroxyacid is salicylic acid, willowbark extract, poly hydroxyacid, tannic acid, hydroxybenzoic acid, juniperic acid, tartaric acid, glycolic acid, lactic acid, citric acid, or a combination thereof.

9. The skincare composition of claim 1 wherein the acid that complexes with colloidal silver is citric acid.

10. The skincare composition of any of claim 1, wherein the colloidal silver is in the form of nanoparticles.

11. A silver textile comprising a textile coated with colloidal silver and an acne treatment ingredient.

12. The silver textile of claim 11, wherein the colloidal silver and/or acne treatment ingredient is in the form of nanoparticles, microparticles, microspheres, micron sized particles, microencapsulated materials, matrices, coated particles, smart particles responsive to environmental stimuli, UV curable particles, low temperature melt particles, or a combination thereof.

13. The silver textile of claim 11, wherein the acne treatment ingredient is selected from benzoyl peroxide, clindamycin, erythromycin, polyhydroxy acid, willowbark extract, salicylic acid, resorcinol, resorcinol monoacetate, niacinamide, sulfur, beta carotene, vitamin A or derivatives thereof, retinol, tretinoin, adapalene, tazarotene, retinyl palmitate, retinyl linoleate, tyrosinase inhibitor, PAR-2 blocker, lignin peroxidase, other ingredients that bind Toll-like 2 receptors, or a combination thereof.

14. The silver textile of claim 11, wherein the textile is a pillowcase, shirt, mask, skin patch, cloth, bed sheet, t-shirt, tank top, pajamas, or a combination thereof.

15. The silver textile of claim 11, wherein the textile comprises every fiber, every second fiber, every third fiber, every fourth fiber, every fifth fiber, every sixth fiber, every seventh fiber, every eighth fiber, every ninth fiber or every tenth fiber coated with the colloidal silver and/or the acne treatment ingredient.

16. The silver textile of claim 11, wherein the silver textile was coated using a technique selected from injection, spray, wash, soaking, binding, depositing, drip, or any other means that achieves uniform application onto the textile.

17. The silver textile of claim 11, wherein the acne treatment ingredient and/or colloidal silver is on the same fiber of the textile.

18. The silver textile of claim 11, wherein the acne treatment ingredient and/or colloidal silver is not on the same fiber of the textile.

19. The silver textile of claim 11, wherein the textile is further coated with an additional metal selected from zinc, copper, nickel, titanium, cobalt, gold, iron, chromium, platinum, plutonium, mercury, nickel, magnesium, cadmium, gadolinium, lithium, tungsten, or a combination thereof.

20. A method of treating acne, the method comprising the steps of applying a skincare composition to an area of skin having acne lesions, or acne-prone skin; and contacting the area of skin with a textile coated with a therapeutically-effective amount of silver, wherein the skincare composition comprises an amphoteric surfactant and/or a zwitterionic surfactant having an isoelectric point of pH 7 or below; a non-ionic surfactant; colloidal silver; an acid to complex with the colloidal silver, where the molar ratio of the acid to the colloidal silver is at least 1:1 (or greater); and one or more hydroxyacid(s).

21. The method of claim 20, wherein the textile is a pillowcase, shirt, mask, skin patch, cloth, bed sheet, t-shirt, tank top, pajamas, or a combination thereof.

22. The method of claim 20, wherein the silver is the form of nanoparticles.

23. The method of claim 20, wherein the silver is colloidal silver.

24. The method of claim 20 further comprising the step of applying a acne treatment composition comprising benzoyl peroxide or a retinoid to the area of skin or to the acne lesions.

25. The method of claim 20, wherein the skincare composition is a cleanser, a toner or a combination thereof.

26. The method of claim 20, wherein the step of applying the skincare composition comprises the steps of cleansing the area of skin with a cleanser having the skincare composition; and applying a toner having the skincare composition onto the area of skin, wherein the hydroxyacid in the skincare composition is salicylic acid.

27. A kit for acne, the kit comprising a silver textile comprising colloidal silver and an acne treatment ingredient; a skincare composition comprising an amphoteric surfactant and/or a zwitterionic surfactant having an isoelectric point of pH 7 or below; a non-ionic surfactant; colloidal silver; an acid to complex with the colloidal silver, where the molar ratio of the acid to the colloidal silver is at least 1:1 (or greater); and one or more hydroxyacid(s); and an acne treatment composition comprising benzoyl peroxide or a retinoid.

28. The kit of claim 27, wherein the acne treatment composition is a cream or gel.

29. The kit of claim 27, wherein the skincare composition is a cleanser, a toner, or a combination thereof.

30. A method of preventing acne on an area of skin that has undergone a resurfacing procedure, the method comprising applying a skincare composition to the area of skin; contacting the area of skin with a textile coated with a therapeutically-effective amount of silver; and applying an acne treatment composition comprising benzoyl peroxide or a retinoid to the area of skin, wherein the skincare composition comprises an amphoteric surfactant and/or a zwitterionic surfactant having an isoelectric point of pH 7 or below; a non-ionic surfactant; colloidal silver; an acid to complex with the colloidal silver, where the molar ratio of the acid to the colloidal silver is at least 1:1 (or greater); and one or more hydroxyacid(s).

31. The method of claim 30, wherein the acne treatment composition is a cream or gel.

32. The method of claim 30, wherein the skincare composition is a cleanser, a toner, or a combination thereof.
